(19) United States
(12) Patent Application Publication
(10) Pub. No.: US 2012/0232287 A1
(43) Pub. Date: Sep. 13, 2012

(54) DETERGENT DISPERSANT AND LUBRICATING OIL COMPOSITION

(75) Inventors: Junya Iwasaki, Sodegaura-shi (JP); Izumi Terada, Sodegaura-shi (JP)

(73) Assignee: IDEMITSU KOSAN CO., LTD., Chiyoda-ku, Tokyo (JP)

(21) Appl. No.: 13/508,831

(22) PCT Filed: Sep. 29, 2010
(86) PCT No.: PCT/JP2010/066928

§ 371 Date: May 9, 2012

(30) Foreign Application Priority Data
Sep. 11, 2009 (JP) 2009-256090

(51) Int. Cl. C07D 403/12; C07C 279/02; C07D 233/50

(52) U.S. Cl. 548/312.7; 548/333.1; 564/241

(57) ABSTRACT

A detergent dispersant contains a nitrogen-containing compound represented by the following formula (1). A lubricating oil composition contains the detergent dispersant.

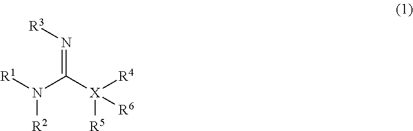

Each symbol in the formula means the same as described in the description.

### TECHNICAL FIELD

The present invention relates to a detergent dispersant and a lubricating oil composition containing the detergent dispersant.

### BACKGROUND ART

Succinimide compounds, hydroxybenzylamine compounds and the like have been known as conventional ashless detergent dispersants. With an excellent dispersibility of dispersing fine particles when such a detergent dispersant is contained in lubricating oil, the detergent dispersant is extensively used as lubricant additives for gasoline engine oil, diesel engine oil, two-cycle engine oil and the like. Moreover, the detergent dispersant is regarded as one of very important additives for lubricants, because the detergent dispersant also has synergistic effects with zinc dialkyl dithiophosphates, metal detergents or the like.

However, the ashless detergent dispersant including succinimide detergent dispersants, hydroxybenzylamine detergent dispersants and the like are insufficient in detergency and stability at high temperatures.

Accordingly, an ashless detergent dispersant formed of a heterocyclic compound having a heterocyclic structure derived from a compound selected from pyridines, pyrroles, pyrimidines, pyrazoles, pyridazines, imidazoles, pyrazines and the like has been proposed (see Patent Literature 1). It is described that this ashless detergent dispersant exhibits excellent high-temperature stability, high-temperature detergency, base value-maintainable property and the like in an application to a gasoline engine or a diesel engine (see paragraph [0010] and the like of the specification).

### CITATION LIST

### Patent Literature(S)

Patent Literature 1 WO2008/153015

### SUMMARY OF THE INVENTION

### Problems to be Solved by the Invention

However, in some cases, even the ashless detergent dispersant described in Patent Literature 1 has a difficulty in exhibiting sufficient performance in high-temperature stability, high-temperature detergency, base value-maintainable property and the like.

An object of the invention is to provide a detergent dispersant having excellent high-temperature stability, high-temperature detergency and base value-maintainable property, and a lubricating oil composition containing the detergent dispersant.

### Means for Solving the Problems

In order to solve the above-mentioned problems, the invention provides a detergent dispersant and a lubricating oil composition containing the detergent dispersant, as described below.

[1] In an aspect of the invention, a detergent dispersant contains a nitrogen-containing compound represented by the following formula (1).

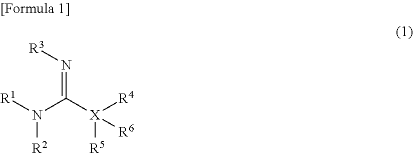

In the formula (1), X represents one of N (nitrogen), O (oxygen), C (carbon) and H (hydrogen); R¹, R², R³, R⁴, R⁵ and R⁶ independently represent hydrogen atoms or hydrocarbon groups that may have at least one substituent or structure selected from amino groups, amide groups, hydroxyl groups, ether groups and carbonyl groups, the hydrocarbon group having 10 to 200 carbon atoms; R¹, R², R³, R⁴, R⁵ and R⁶ may be mutually the same or different, but all of those are not hydrogen at the same time; R¹ and R², R⁴ and R⁵, and R² and R⁴ may respectively form cyclic structures; and R⁶ is not present when X is N, R⁵ and R⁶ are not present when X is O, and R⁴, R⁵ and R⁶ are not present when X is H.

[2] In the detergent dispersant according to the above aspect of the invention, X is N (nitrogen).

[3] In the detergent dispersant according to the above aspect of the invention, the nitrogen-containing compound represented by the formula (1) has a number average molecular mass of 120 to 5000.

[4] In the detergent dispersant according to the above aspect of the invention, the nitrogen-containing compound represented by the formula (1) further contains boron.

[5] In another aspect of the invention, a lubricating oil composition contains the detergent dispersant according to the above aspect of the invention.

[6] In the lubricating oil composition according to the above aspect of the invention, the lubricating oil composition is used for an internal combustion engine.

### Effect(s) of the Invention

A lubricating oil composition containing a detergent dispersant of the invention exhibits excellent high-temperature stability, high-temperature detergency and base value-maintainable property. Accordingly, the lubricating oil composition is particularly suitable for an internal engine such as a gasoline engine, a diesel engine or a two-cycle engine

### DESCRIPTION OF EMBODIMENT(S)

In an exemplary embodiment, a detergent dispersant contains a nitrogen-containing compound represented by the following formula (1).

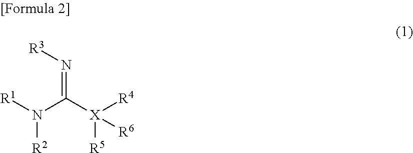

In the formula (1), X represents one of N (nitrogen), O (oxygen), C (carbon) and H (hydrogen). R¹, R², R³, R⁴, R⁵ and R⁶ independently represent hydrogen atoms or hydrocarbon groups. The hydrocarbon group may have at least one substituent or structure selected from amino groups, amide groups, hydroxyl groups, ether groups and carbonyl groups. The hydrocarbon group has 10 to 200 carbon atoms. When the carbon atoms of the hydrocarbon group are less than 10 or more than 200, detergent dispersibility may be lowered.

R¹, R², R³, R⁴, R⁵ and R⁶ may be mutually the same or different, but all of those are not hydrogen at the same time. R¹ and R², R⁴ and R⁵, and R² and R⁴ may respectively form cyclic structures. The cyclic structure may be saturated or unsaturated. Carbon atoms in the cyclic structure are preferably in a range of 2 to 12.

In the above formula (1), R⁶ is not present when X is N, R⁵ and R⁶ are not present when X is O, and R⁴, R⁵ and R⁶ are not present when X is H.

The nitrogen-containing compound represented by the formula (1) preferably has a number average molecular mass of 120 to 5000, more preferably of 200 to 4000. When the number average molecular mass is less than 120 or more than 5000, detergent dispersibility may be lowered.

The nitrogen-containing compound represented by the formula (1) includes a group of compounds called guanidine compounds or amidine compounds.

The guanidine compound may be exemplified by a nitrogen-containing compound represented by the following formulae (2) and (6). C₁₀ and C₁₂ represent hydrocarbon groups and carbon atoms thereof in the formulae. Properties of the detergent dispersant containing such a nitrogen-containing compound will be described in detail in the following Examples.

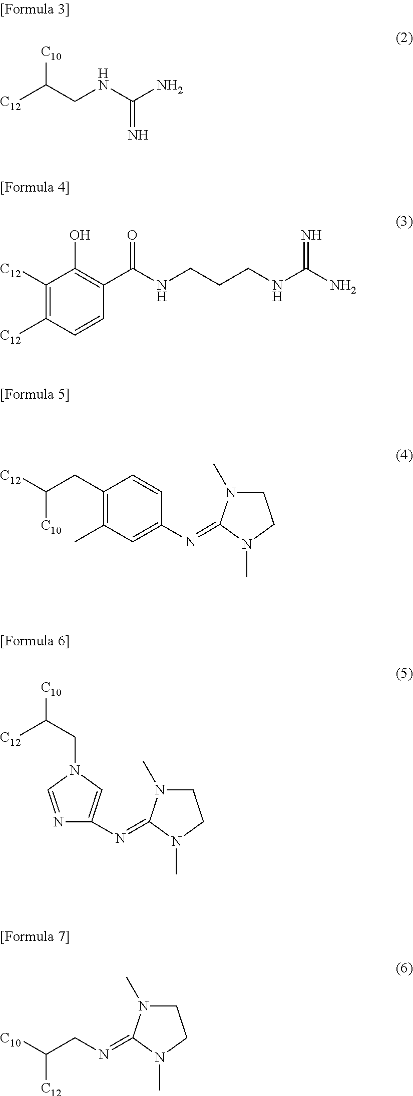

The amidine compound may be exemplified by various nitrogen-containing compounds as shown below. R and R′ represent organic groups such as hydrocarbon groups in the following formulae.

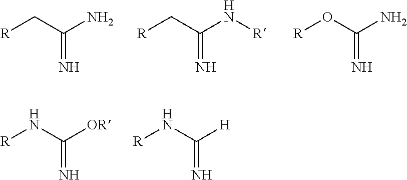

The nitrogen-containing compound represented by the above formula (1) is exemplarily producible as follows.

The nitrogen-containing compound represented by the formula (1) is preferably obtainable by reacting (a) a nitrogen-containing portion of the nitrogen-containing compound represented by the formula (1) (e.g., a nitrogen-containing portion of compounds or derivatives thereof having guanidine skeleton or amidine skeleton) with (b) a nitrogen-containing portion of compounds having alkyl groups, alkenyl groups, cycloalkyl groups, or aryl groups having 10 to 200 carbon atoms that only contains carbon atoms, or further contains an oxygen atom or a nitrogen atom, preferably at a ratio (a):(b)=1:5 to 5:1 by mol, more preferably at a ratio (a):(b)=1:2 to 2:1 by mol. The reaction product may be used without any treatment or after refinement by column chromatography, distillation, recrystallization or the like.

Further, the nitrogen-containing compound represented by the formula (1) may contain boron. For instance, the nitrogen-containing compound further containing boron is obtainable by reacting the nitrogen-containing compound obtained as described above with a boron-containing compound preferably at a ratio of 1:0.01 to 1:10 by mol, more preferably at a ratio of 1:0.05 to 1:5 by mol. The reaction between the nitrogen-containing compound and the boron-containing compound is performed preferably at a temperature of 50 degrees C. to 250 degrees C., more preferably at a temperature of 100 degrees C. to 200 degrees C. In performing the reaction, a solvent such as an organic solvent (e.g., a hydrocarbon oil) may be used. Examples of the boron compound are boric oxide, boron halogenide, boric acid, boric anhydride and borate ester.

Specifically, the nitrogen-containing compound of the formula (1) is exemplarily produced by reacting (a) 1-amidino pyrazole hydrochloride with (b) 2-decyltetradecyl amine in a range of −50 degrees C. to 200 degrees C., preferably in a range of −10 degrees C. to 150 degrees C. The reaction may be performed without a catalyst or under the presence of a catalyst. Further, in performing the reaction, an organic solvent such as hexane, toluene, xylene, THF and DMF may be used.

The detergent dispersant of the invention containing the nitrogen-containing compound represented by the formula (1) is blended in lubricant base oil and is preferably used as a lubricating oil composition. For instance, the nitrogen-containing compound represented by the formula (1) may be blended in lubricant base oil as a detergent dispersant without any treatment. Alternatively, the nitrogen-containing compound may be blended in lubricant base oil after being mixed with another additive (i.e., as an additive package).

Specifically, it is preferable that the detergent dispersant of the invention is blended in lubricant base oil at a rate in a range of 0.01 mass % to 20 mass % based on the total amount of the composition in terms of the nitrogen-containing compound represented by the formula (1), thereby providing a lubricating oil composition. A content of the detergent dispersant is preferably in a range of 0.05 mass % to 15 mass %, more preferably in a range of 0.1 mass % to 10 mass %. When the content of the detergent dispersant is less than 0.01 mass %, sufficient effects of the invention may not be obtained. When the content of the detergent dispersant is more than 20 mass %, enhancement of effects may not be expected.

Both mineral oil and synthetic oil are usable as the lubricant base oil. Any lubricating oil such as paraffinic mineral oil, naphthenic mineral oil and aromatic mineral oil is usable as the mineral oil. Moreover, a mineral oil produced by any refining method such as solvent purification, hydrogenation purification or hydrocracking is also usable. Examples of the synthetic oil include polyphenylether, alkylbenzene, alkyl naphthalene, an ester oil, a glycol-synthetic oil, and a polyolefin-synthetic oil One of the mineral oil and the synthetic oil may be used alone, or a mixture thereof may be used.

Because of the aforementioned effects, the lubricating oil composition containing the detergent dispersant of the invention is also suitable for gear oil, bearing oil, transmission oil, shock absorber oil, and industrial lubricant oil in addition to lubricant oil suitable for an internal combustion engine such as a gasoline engine, a diesel engine and a two-cycle engine.

The detergent dispersant of the invention provides the same effects not only as a lubricating oil additive but also as a fuel oil additive. For instance, the fuel oil composition obtained by blending the detergent dispersant of the invention with a fuel oil (e.g., hydrocarbon) exhibits excellent effects on preventing a contaminant from being attached to a carburetor of an internal combustion engine and removing the attached matter.

Note that an antioxidant, an antiwear agent, other detergent dispersants, a viscosity index improver, a pour point depressant or other additives, which are typically blended in a lubricating oil, may be used together as long as the effects of the invention is not impeded.

Examples of the antioxidant include a phenolic antioxidant, an amine antioxidant, a molybdenum-amine-complex antioxidant and a sulfuric antioxidant. As the phenolic antioxidant, a bisphenol antioxidant and an ester group-containing phenolic antioxidant are preferable. As the amine antioxidant, a dialkyl diphenylamine antioxidant and a naphthylamine antioxidant are preferable. The content of the antioxidant is preferably in a range of approximately 0.1 mass % to 5 mass % based on the total amount of the lubricating oil composition, more preferably in a range of approximately 0.1 mass % to 3 mass %.

Examples of the antiwear agent include: a sulfur-containing compound such as zinc dithiophosphate, zinc phosphate, zinc dithiocarbamate, molybdenum dithiocarbamate, molybdenum dithiophosphate, disulfides, sulfurized olefins, sulfurized fats and oils, sulfurized esters, thiocarbonates, thiocarbamates and polysulfides; a phosphorus-containing compound such as phosphite esters, phosphate esters, phosphonate esters and amine salts or metal salts thereof; and a sulfur and phosphorus-containing antiwear agent such as thiophosphite esters, thiophosphate esters, thiophosphonate esters and amine salts or metal salts thereof. When the antiwear agent is blended, a content of the antiwear agent is preferably in a range of approximately 0.05 mass % to 5 mass % based on the total amount of the composition.

As other detergent dispersant, an ashless dispersant and a metal detergent are usable. As the ashless dispersant, any ashless dispersant typically used for a lubricating oil can be blended. Examples of the ashless dispersant include a mono-type alkenyl- or alkyl-succinimide compound, a bis-type alkenyl- or alkyl-succinimide compound, and polyamine. In addition to the alkenyl- or alkyl-succinimide compound, a boron derivative thereof, or a compound obtained by modifying the alkenyl- or alkyl-succinimide compound or the boron derivative by an organic acid may be used. The boron derivative of the alkenyl- or alkyl-succinimide compound may be produced by a typical method for use. For instance, the boron derivative of the alkenyl- or alkyl-succinimide compound is obtainable by: reacting polyolefin with maleic anhydride to provide alkenyl succinic anhydride; and reacting the alkenyl succinic anhydride with polyamine and an intermediate body obtained by reacting a boron compound such as a boric oxide, a boron halogenide, boric acid, boric anhydride, a borate ester, an ammonium salt of boric acid for imidization.

A boron content in the boron derivative is subject to no limitation, but is typically in a range of 0.05 mass % to 5 mass %, preferably in a range of 0.1 mass % to 3 mass %.

A content of the mono-type succinimide compound or the bis-type succinimide compound is preferably in a range of 0.5 mass % to 15 mass % based on the total amount of the composition, more preferably in a range of 1 mass % to 10 mass %. When the content is less than 0.5 mass %, the effects are difficult to be exhibited. When the content is more than 15 mass %, effects relative to the added content cannot be obtained. As long as the content described above is used, one of the succinimide compounds may be used alone or a combination of two or more thereof may be used.

The metal detergent may be any alkyl earth metal detergent usable in the lubricating oil. For example, any one of alkaline earth metal sulfonate, alkaline earth metal phenate and alkaline earth metal salicylate and a mixture of two or more selected therefrom are usable. The metal detergent may be a neutral salt, a basic salt, an overbased salt or a mixture thereof. Particularly, a mixture of at least one of an overbased salicylate, an overbased phenate and an overbased sulfonate with an neutral sulfonate is preferable in terms of detergency and wear resistance. The metal detergent is typically commercially available in dilution by a light lubricant base oil and the like and is easily available. In general, a metal content of the metal detergent is preferably in a range of 1 mass % to 20 mass %, more preferably in a range of 2 mass % to 16 mass %. A content of the metal detergent is preferably in a range of 0.01 mass % to 20 mass % based on the total amount of the composition, more preferably in a range of 0.1 mass % to 10 mass %. When the content is less than 0.01 mass %, the effects are difficult to be exhibited. When the content is more than 20 mass %, effects relative to the added content cannot be obtained. As long as the content of the metal detergent is within the above-mentioned range, one of the metal detergents may be used alone or a combination of two or more thereof may be used.

Examples of the viscosity index improver include polymethacrylate, dispersed polymethacrylate, an olefin copolymer (e.g., an ethylene-propylene copolymer), a dispersed olefin copolymer, and a styrene copolymer (e.g., a styrene-diene copolymer and a styrene-isoprene copolymer). In view of blending effects, a content of the viscosity index improver is in a range of approximately 0.5 mass % to 15 mass % based on the total amount of the composition, preferably in a range of 1 mass % to 10 mass %.

The pour point depressant is exemplified by polymethacrylate having a mass average molecular mass of approximately 5000 to 50000. In view of blending effects, a content of the pour point depressant is in a range of 0.1 mass % to 2 mass % based on the total amount of the composition, preferably in a range of 0.1 mass % to 1 mass %.

Examples of other additives include a rust inhibitor, a metal deactivator, an antifoaming agent, and a surfactant.

### EXAMPLES

The invention will be further described in detail below with reference to Examples and Comparatives, which by no means limit scope of the invention.

### Manufacturing Example 1

To a 500-ml flask, 20.0 g (0.057 mol) of 2-decyltetradecyl amine, 150 ml of DMF and 90 ml of toluene were put into a 500-ml flask. 9.1 g (0.062 mol) of 1-amidino pyrazole hydrochloride and 16.2 ml of diisopropylethylamine were added thereto to react for eight hours at room temperature. After the reaction, a solvent was distilled away from the reaction solution. The residue was dissolved in 300 ml of hexane and washed by 2 M aqueous sodium hydroxide and water. The washed solution was dried over magnesium sulfate and hexane was distilled away. Further, the reaction product was deinonized using an ion exchange resin (Amberlite) to obtain a target. The resulting target was the nitrogen-containing compound (2-decyltetradecyl guanidine) represented by the above formula (2), the yield of which was 23.7 g.

### Manufacturing Example 2

40 g (0.082 mol) of methyl didodecyl salicylic acid, 12.1 g (0.164 mol) of diaminopropane and 10 ml of toluene were put into a 200-ml flask, and reacted at 30 degrees C. for 36 hours. After the reaction, the reaction mixture was further added with 200 ml of hexane, and washed by water. The washed solution was dried over magnesium sulfate and hexane was distilled away. Next, 20 g (0.038 mol) of the obtained reaction product, 90 ml of DMF and 60 ml of toluene were put to a 300-ml flask. 6.2 g (0.042 mol) of 1-amidino pyrazole hydrochloride and 10.8 ml of diisopropylethylamine were added thereto to react at room temperature for eight hours. After the solvent was distilled away, the residue was dissolved in 300 ml of hexane and washed by 2 M aqueous sodium hydroxide and water. The washed solution was dried over magnesium sulfate and hexane was distilled away. Further, the reaction product was deinonized using an ion exchange resin (Amberlite) to obtain a target. The resulting target was the nitrogen-containing compound represented by the above formula (3), the yield of which was 22.1 g.

### Manufacturing Example 3

3.0 g (0.007 mol) of 2-decyl tetradecyl methyl aniline, 1.24 g (0.007 mol) of 2-chloro-1,3-dimethyl imidazolium chloride, 20 ml of acetonitrile and 30 ml of toluene were put into a 200-ml flask and cooled to 0 degree C. After 1.1 ml of triethylamine was added and stirred for 30 minutes, the temperature of the reaction product was raised and reacted for eight hours while being refluxed. The reaction product was dissolved in 300 ml of hexane and washed by 12.5 M aqueous sodium hydroxide and water. The washed solution was dried over magnesium sulfate and hexane was distilled away. The resulting target was the nitrogen-containing compound represented by the above formula (4), the yield of which was 3.2 g.

### Manufacturing Example 4

The same process as in Manufacturing Example 3 was repeated. In Manufacturing Example 4, however, used was 3.0 g (0.007 mol) of 1-(2-decyltetradecyl)-4-aminoimidazole in place of 2-decyl tetradecyl methyl aniline. The resulting target was the nitrogen-containing compound represented by the above formula (5), the yield of which was 3.1 g.

### Manufacturing Example 5

The same process as in Manufacturing Example 3 was repeated. In Manufacturing Example 5, however, used was 2.5 g (0.007 mol) of 2-decyltetradecyl amine in place of 2-decyl tetradecyl methyl aniline. The resulting target was the nitrogen-containing compound represented by the above formula (6), the yield of which was 2.6 g.

### Manufacturing Example 6

20 g (0.051 mol) of 2-decyl tetradecyl guanidine manufactured in Manufacturing Example 1 and 1.18 g (0.019 mmol) of boric acid were put into a 300-ml flask and reacted at 150 degrees C. for four hours in nitrogen gas stream with stirring. Subsequently, generated water was distilled away under reduced pressure at 150 degrees C. and filtered to obtain 20.5 g of a boron-and-nitrogen-containing compound as a target.

### Manufacturing Comparative 1

1,100 g (Mw: 987) of Polybutene, 6.4 g (0.021 mol) of cetyl bromide and 115 g (1.2 mol) of maleic acid anhydride were put into a 2-L autoclave, purged with nitrogen, and reacted at 240 degrees C. for five hours. After the temperature was cooled to 215 degrees C., the non-reacted maleic acid anhydride and cetyl bromide were removed through distillation under reduced pressure, further cooled to 140 degrees C., and then filtered. The yield of the resulting polybutenyl succinic anhydride was 1,100 g. The obtained polybutenyl succinic anhydride (500 g) was put into a 2-L separable flask, along with 64 g (0.34 mol) of tetraethylenepentamine (TEPA) and 300 g of 150-neutral fraction mineral oil thereinto, and reacted with each other in nitrogen gas stream at 150 degrees C. for two hours. Next, after the resulting product was heated up to 200 degrees C., the non-reacted TEPA and generated water were removed through distillation under reduced pressure, cooled to 140 degrees C., and filtered. The yield of the resulting polybutenyl succinimide was 790 g.

### Manufacturing Comparative 2

The same process as in Manufacturing Comparative 1 was repeated. In Manufacturing Comparative 2, however, used was 915 g (Mw: 800) of polybutene in place of polybutene (Mw: 987). The yield of the obtained polybutenyl succinic anhydride was 940 g. Next, in the same manner as in Manufacturing Comparative 1, 500 g of the polybutenyl succinic anhydride was reacted with 76 g (0.40 mol) of tetraethylenepentamine (TEPA) and 300 g of 150-neutral fraction mineral oil. The yield of the resulting polybutenyl succinimide was 810 g.

### Manufacturing Comparative 3

The same process as in Manufacturing Comparative 1 was repeated. In Manufacturing Comparative 3, however, used was 890 g (Mw: 445) of polybutene in place of polybutene (Mw: 987) and 11 g (0.036 mol) of cetyl bromide and 397 g (2.1 mol) of maleic acid anhydride. The yield of the resulting polybutenyl succinic anhydride was 990 g. Next, in the same manner as in Manufacturing Comparative 1, (500 g) of the polybutenyl succinic anhydride was reacted with 88 g (0.60 mol) of triethylenetetramine (TETA) and 300 g of 150-neutral fraction mineral oil. The yield of the resulting polybutenyl succinimide was 820 g.

### Examples 1 to 6 and Comparatives 1 to 3

Each of the nitrogen-containing compounds obtained in Examples and the succinimide compounds in Comparatives was blended in a mineral oil of 500-neutral fraction mineral oil at 10 mass % based on the total amount of the composition to prepare a lubricating oil composition (test oil) for evaluation.

A hot tube test and a test for base value-maintainable property were conducted on these test oils and performance of each of the blended detergent dispersants therein was evaluated. Results are shown in Table 1.

### Hot Tube Test

A test oil of 0.3 ml/hr and air of 10 ml/min were kept on flowing in a glass tube having 2-mm inner diameter for 16 hours while a temperature of the glass tube was kept at 250 degrees C. Lacquer (deposits) attached inside the glass tube was compared with a color sample. When the lacquer was transparent, 10 points was given as a score and when the lacquer was black, no point was given. At the same time, the mass of the lacquer attached inside the glass tube was also measured. The results show that the larger the score becomes or the smaller the mass of the attached lacquer becomes, the higher the performance of the lubricating oil composition is.

### Base Value-Maintainable Property Test

The test oil was collected after the hot tube test described above. A base value of the test oil was measured according to a hydrochloric acid method. The measured base value was compared with a base value before the test to evaluate a base value-maintainable property based on a residual base-value rate (%). A higher residual base-value rate shows a higher base value-maintainable property. A calculation equation is as follows.

residual base-value rate=(residual base value/initial base value)×100(%)

TABLE 1

| | | | | Base Value-Maintainable | |
|---|---|---|---|---|---|
| | | | | Property Test | |
| | | Hot Tube Test | | initial | residual |
| | detergent | | deposits | base value | base value |
| | dispersant | score | (mg) | (mgKOH/g) | rate (%) |
| Example 1 | Manufacturing | 8 | 1.4 | 12.5 | 46 |
| | Ex. 1 | | | | |
| Example 2 | Manufacturing | 10 | 0 | 5.8 | 23 |
| | Ex. 2 | | | | |
| Example 3 | Manufacturing | 8 | 1.3 | 8.4 | 28 |
| | Ex. 3 | | | | |
| Example 4 | Manufacturing | 10 | 0 | 3.8 | 60 |
| | Ex. 4 | | | | |
| Example 5 | Manufacturing | 9 | 3 | 14.2 | 20 |
| | Ex. 5 | | | | |
| Example 6 | Manufacturing | 10 | 0 | 8.9 | 42 |
| | Ex. 6 | | | | |
| Comp. 1 | Manufacturing | 0 | 45 | 7.7 | 2 |
| | Comp. 1 | | | | |
| Comp. 2 | Manufacturing | 0 | 48 | 8.5 | 1 |
| | Comp. 2 | | | | |
| Comp. 3 | Manufacturing | 0 | 57 | 9.8 | 2 |
| | Comp. 3 | | | | |

Evaluation Results The result in Table 1 shows that all the test oils of Examples 1 to 6 containing the detergent dispersant of the invention exhibit excellent high-temperature stability, high-temperature detergency and base value-maintainable property. On the other hand, the test oils of Comparatives 1 to 3, which contain an all-purpose ashless detergent dispersant, are inferior to the test oils of Examples in terms of all of high-temperature stability, high-temperature detergency and base value-maintainable property.

### INDUSTRIAL APPLICABILITY

The invention relates to a detergent dispersant and a lubricating oil composition containing the detergent dispersant.

1. A detergent dispersant containing a nitrogen-containing compound represented by the following formula (1), 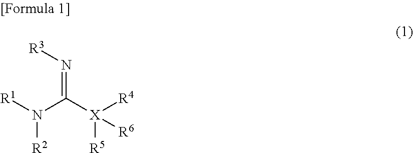
where: X represents one of N (nitrogen), O (oxygen), C (carbon) and H (hydrogen); R¹, R², R³, R⁴, R⁵ and R⁶ independently represent hydrogen atoms or hydrocarbon groups that are allowed to have at least one substituent or structure selected from amino groups, amide groups, hydroxyl groups, ether groups and carbonyl groups, the hydrocarbon group having 10 to 200 carbon atoms; R¹, R², R³, R⁴, R⁵ and R⁶ are allowed to be mutually the same or different, but all of those are not hydrogen at the same time; R¹ and R², R⁴ and R⁵, and R² and R⁴ are allowed to respectively form cyclic structures; and R⁶ is not present when X is N, R⁵ and R⁶ are not present when X is O, and R⁴, R⁵ and R⁶ are not present when X is H.

2. The detergent dispersant according to claim 1, wherein X is N (nitrogen).

3. The detergent dispersant according to claim 1 or 2, wherein
the nitrogen-containing compound represented by the formula (1) has a number average molecular mass of 120 to 5000.

4. The detergent dispersant according to any one of claims 1 to 3, wherein
the nitrogen-containing compound represented by the formula (1) further contains boron.

5. A lubricating oil composition comprising the detergent dispersant according to any one of claims 1 to 4.

6. The lubricating oil composition according to claim 5, wherein the lubricating oil composition is used for an internal combustion engine.
